(19) United States
(12) Patent Application Publication
(10) Pub. No.: US 2016/0015271 A1
(43) Pub. Date: Jan. 21, 2016

(54) PALPATION DIAGNOSTIC DEVICE

(71) Applicant: National Cheng Kung University, Tainan City (TW)

(72) Inventors: Wei-Chih WANG, Tainan City (TW); Chih-Han CHANG, Tainan City (TW); Fong-Chin SU, Tainan City (TW); David LINDERS, Cambridge, MA (US)

(21) Appl. No.: 14/335,308

(22) Filed: Jul. 18, 2014

(51) Int. Cl. A61B 5/00

(52) U.S. Cl. CPC A61B 5/0053; A61B 5/6826; A61B 5/6839; A61B 5/0004; A61B 2562/0247; A61B 2562/0266; A61B 2560/0412; A61B 2562/164; A61B 2562/223

(57) ABSTRACT

The present invention relates to a palpation diagnostic device, which comprises an optical pressure sensor embedded in a holder; wherein the optical pressure sensor is an optical fiber sensor, or a micro-fabricated waveguide sensor to be disposed on a finger or a palm; and the optical pressure sensor is configured to receive an optical signal whose intensity is attenuated when a force is applied on the optical pressure sensors. Therefore, the palpation diagnostic device of the present invention can provide high sensing sensitivity by attenuating the intensity of the optical signal in the optical pressure sensors which a force is applied on, so it can provide precise and immediate information based on quantitative feedback for the users.

### BACKGROUND OF THE INVENTION

1. Field of the Invention

The present invention relates to a palpation diagnostic device, and more particularly, to a pressure change sensing device adapted for medical treatment.

2. Description of Related Art

Patient diagnosis and treatment frequently involves the clinician placing hands upon the patient and manually manipulating joints and muscles. Touch and pressure frequently play a significant role in diagnosing and treating disease. For example, a surgeon learns through experience what a healthy liver feels like and how it differs from a diseased or damaged organ. A chiropractor relies on previous work and memory to assess and treat a patient with a painful spine. A physical therapist sets a shoulder or stretches a muscle with a specific force that will help heal an injury without causing harm. In all of these cases, clinicians apply forces that need to be sensitive and accurate, and they currently do so without quantitative feedback. The results of examinations and treatments in these important fields cannot be practically measured or recorded. As a result, trial and error still plays a major role, and further leads to misdiagnosis and undesirable outcomes. As such, manual healthcare providers are looking to adapt leading-edge technologies to improve diagnoses and treatments.

Quantifying manual force application has been accomplished theoretically, through inverse dynamics, and via direct measurement. Measurement of the forces clinicians apply to their patients has been accomplished using instrumented tools, gloves, and tables. Together, these measurement systems have improved the knowledge base for physical medicine and individual patient care.

For example, instrumented tables and couches have been developed to measure the forces the clinician applies to the patients. These have resulted in vital data but are limited to the application of the forces through the patient to the table. However, the current tools and associated data have resulted in significant improvements in treatment, but regrettably, these varied instrumented tools and tables can only produce tool-specific data, and cannot directly measure at the clinician's point of force application.

A few fingertip tactile sensors using optical sensors have also been reported in robotics related research. A commercially available tactile sensor from Tactile Robotic Systems (Sunnydale, Calif.) operates by detecting the amount of light coupling between source and detector fiber parts. An applied force causes relative motion between the fibers, resulting in light attenuation. However, this design requires very precise and rigid support. It is also sensitive to vibration. Optical touch sensors based on total internal reflection and light scattering were also demonstrated, but possess limited sensitivity and repeatability reliant on the cleanness of the fingertip.

In Chinese medicine, examination methods have been based on qualitative examination instead of a quantitative one for a long time. These exams include inspection by listening, smelling, inquiring and palpation. Among all the exams, the most common practice is the latter, in which the illness is usually detected by a sense of touch. Other methods involve long years of training in recognizing patterns of disease or scientifically unexplained “Qi” in explaining how blood, neurons, and body fluids flow. Although some of these phenomena have been studied and quantified somewhat in a scientific way, it is our intention to create a tool to provide quantitative feedback to the clinician so that a more consistent diagnosis can be made. Here we propose an optical force sensor in a patch configuration to assist the palpation diagnosis.

To solve the above-mentioned problem, persistent research and experiments for a “palpation diagnostic device” has been undertaken, eventually resulting in accomplishment of the present invention.

### SUMMARY OF THE INVENTION

The object of the present invention is to provide a device for sensing pressure, and to provide information based on quantitative feedback for the users so that a more consistent diagnosis can be made.

To achieve the object, the palpation diagnostic device of the present invention includes an optical pressure sensor embedded in a holder; wherein the optical pressure sensor is an optical fiber sensor, or a micro-fabricated waveguide sensor to be disposed on a finger or a palm; and the optical pressure sensor is configured to receive an optical signal whose intensity is attenuated when an force is applied on the optical pressure sensors.

Therefore, the palpation diagnostic device of the present invention can provide high sensing sensitivity by attenuating the intensity of the optical signal in the optical pressure sensors which a force is applied on, so it can provide precise and immediate information based on quantitative feedback for the users.

In the palpation diagnostic device of the present invention, the optical pressure sensor is deformed through the force applied on the holder such that the intensity of the optical signal in the optical pressure sensor is attenuated in response to the applied force.

In the palpation diagnostic device of the present invention, the holder is a pad which includes a first polymer applicator patch and a second polymer applicator patch.

In addition, the optical pressure sensor is the optical fiber sensor which comprises a sensing fiber embedded in between the first polymer patch and the second polymer patch.

Moreover, the sensing fiber is slightly bent in accordance with the applied force applied on the first polymer patch, the intensity of the optical signal in the sensing fiber is attenuated by micro-bend sensing fiber, and the attenuated intensity determines the value of the applied force on the first polymer patch based on an attenuation of the intensity of the optical signal.

In the palpation diagnostic device of the present invention, the first polymer patch comprises: a plurality of first teeth disposed on a surface of the first polymer patch, and a plurality of second teeth disposed on a surface of the second polymer patch and engaged with the plurality of corresponding first teeth, and the sensing fiber is in a series of corrugated shape by means of the plurality of first teeth and the plurality of second teeth.

In the palpation diagnostic device of the present invention, the sensing fiber is covered by an elastomer between the first polymer patch and the second polymer patch.

In the palpation diagnostic device of the present invention, the first polymer patch and the second polymer patch are selected from a group consisting of a polymer, a plastic, a silicone rubber, polydimethylsiloxane (PDMS), elastomeric polymer containing polydimethylsiloxane (PDMS), or the combinations thereof.

The palpation diagnostic device of the present invention can further comprise a control device, wherein the control device is electrically coupled to the optical pressure sensor.

In addition, the control device can optionally comprise a control module, a light source, and a detector; the control module is electrically coupled to the light source and detector, the light source and the detector is electrically coupled to the sensing fiber and the reference fiber respectively; and the light source is arranged to emit the optical signal to the sensing fiber and the reference fiber, the detectors are arranged to receive the optical signal from the sensing fiber and the reference fiber, and the control module is arranged to receive the optical signal from the detectors and process the optical signal therein.

Another preferred embodiment of the present invention provides a pressure sensing apparatus, comprising: an optical pressure sensor embedded in a holder; wherein the optical pressure sensor is an optical fiber sensor or a micro-fabricated waveguide sensor; the optical pressure sensor is provided with a phase modulation, a micro-bend loss structure, or a macro-bend loss structure to perform quantitative sensing.

Therefore, the pressure sensing apparatus of the present invention can provide precise and immediate information based on quantitative feedback for the users by changing the optical characteristic in the optical pressure sensor.

In the palpation diagnostic device of the present invention, the holder is preferred to be a pad which comprises a first polymer patch and a second polymer patch.

In a preferred palpation diagnostic device of the present invention, the optical pressure sensor is provided with the Michelson interferometer configuration which comprises: a 2×2 coupler, a first sensing arm, a second sensing arm, a photodetector, and a light source; wherein the 2×2 coupler is coupled to the first sensing arm, second sensing arm, a photodetector, and a light source respectively; an optical signal emitted from the light source becomes two input optical signals with same light intensity through the 2×2 coupler, the two input optical signals pass through the first sensing arm and the second sensing arm to both endpoints therein so as to become two reflected optical signals; and when the two reflected optical signals pass through the first sensing arm and the second sensing arm respectively to the photodetector by the 2×2 coupler, there is a phase shift between the two reflected optical signals so that it shall be coupled to form an interference pattern.

In addition, the phase shift will be changed in accordance with the bending level of the first sensing arm and second sensing arm when the first sensing arm and second sensing arm are bent by the applied force. Wherein, the pressure sensing apparatus is operated in a linear region when the phase shift imposed by the applied force is lower than π/2, and the light intensity of the interference pattern will change in accordance with the phase shift. Moreover, the pressure sensing apparatus is operated in a nonlinear region and is provided with the plurality of interference pattern of interference fringe when the phase shift imposed by the applied force is upper than π/2.

In the palpation diagnostic device of the present invention, the sensing arms are embedded in between the first polymer patch and the second polymer patch.

In the palpation diagnostic device of the present invention, the sensing arms are covered by the elastomer between the first polymer patch and the second polymer patch.

In the palpation diagnostic device of the present invention, the first polymer patch comprises a plurality of first teeth disposed on a surface of the first polymer patch and a plurality of second teeth disposed on a surface of the second polymer patch and engaged with the plurality of corresponding first teeth, and the sensing fiber is a series of corrugated shape by means of the plurality of first teeth and the plurality of second teeth.

Other objects, advantages, and novel features of the invention will become more apparent from the following detailed description when taken in conjunction with the accompanying drawings.

### BRIEF DESCRIPTION OF THE DRAWINGS

FIG. 1 is a schematic diagram of a palpation diagnostic device according to a preferred embodiment of the present invention;

FIG. 2 is a using schematic diagram of a palpation diagnostic device according to a preferred embodiment of the present invention;

FIG. 3 is a systematic diagram of a palpation diagnostic device according to a preferred embodiment of the present invention;

FIG. 4 is a connecting schematic diagram of a sensing fiber and reference fiber according to a preferred embodiment of the present invention;

FIG. 5 illustrates a usage state of a palpation diagnostic device and an analysis system according to a preferred embodiment of the present invention;

FIG. 6 is a side sectional view of an optical pressure sensor according to a preferred embodiment of the present invention;

FIG. 7 is a schematic diagram of an optical pressure sensor according to a preferred embodiment of the present invention;

FIG. 8 illustrates a micro-bend sensing fiber according to a preferred embodiment of the present invention;

FIG. 9 is a schematic diagram of a polymer patch manufacturing device according to a preferred embodiment of the present invention;

FIG. 10 is a schematic diagram of a pressure sensing apparatus according to alternate preferred embodiment of the present invention; and

FIG. 11 is a schematic diagram of a pressure sensing apparatus according to another alternate preferred embodiment of the present invention.

### DETAILED DESCRIPTION OF THE PREFERRED EMBODIMENT

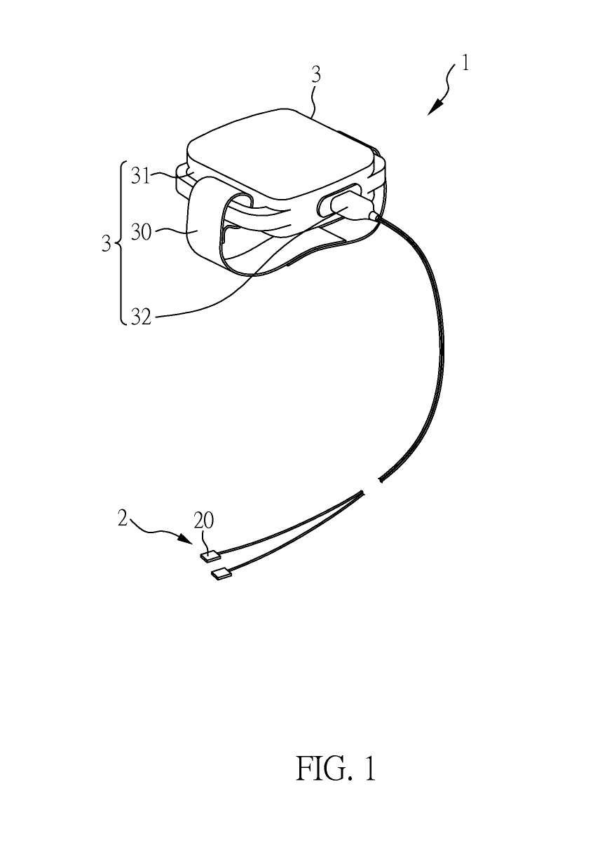

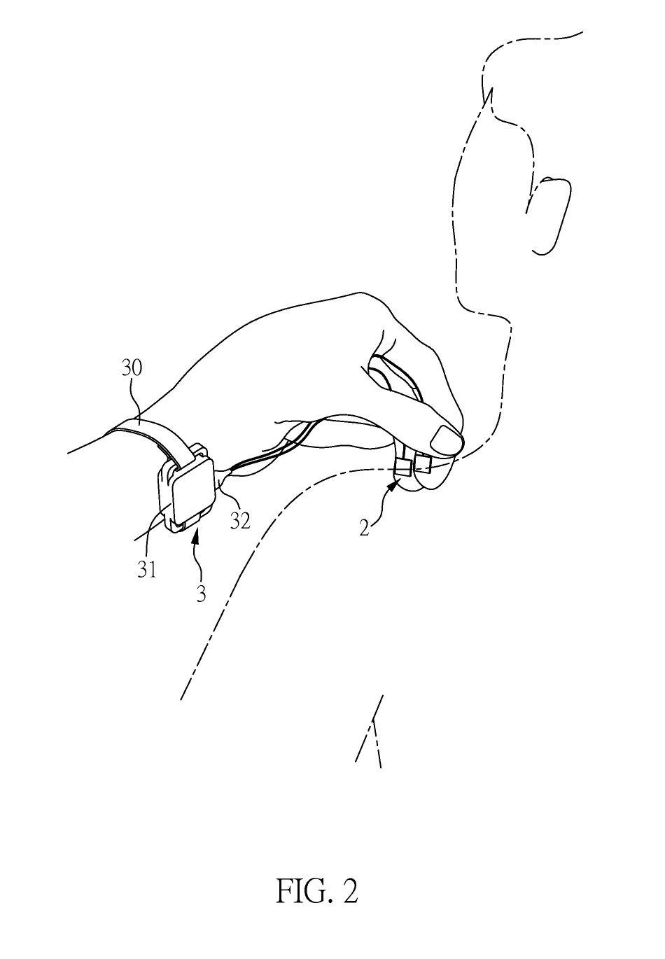

With reference to FIG. 1 and FIG. 2, a schematic diagram and a using schematic diagram of a palpation diagnostic device according to a preferred embodiment of the present invention. As shown in FIG. 1, a palpation diagnostic device 1 comprising a plurality of optical pressure sensor 2 and control device 3. Every optical pressure sensor 2 is embedded in a holder 20 which is a pad or a glove, and can be an optical fiber sensor or a micro-fabricated waveguide sensor to be disposed on a finger or a palm. In the present embodiment, the holder 20 is a pad. The control device 3 is electrically coupled to the optical pressure sensor 10, including a wrist cuff 30, a housing 31 and a connector 32.

Then, with reference to FIG. 2, the wrist cuff 30 is connected to the connector 32 so that the palpation diagnostic device 1 can be sleeved through the user's wrist by using the wrist cuff 30, and the connector 32 is electrically coupled to electronic components in the housing 31 and the optical pressure sensor 2 respectively. In the present embodiment, every optical pressure sensor 2 is an optical fiber sensor, being disposed on a user's finger and including a sensing fiber 21 which is embedded in a holder at an end thereof and is electrically coupled to the connector 32 at the other end thereof. In addition, the optical pressure sensor 2 is configured to receive an optical signal whose intensity is attenuated when a force is applied on the optical pressure sensors. A reference fiber 25 is also embedded in the same holder at an end thereof and is electrically coupled to the connector 32 at the other end thereof. The reference fiber 25 on the other hand is not affect by the force applied on the optical pressure sensor even though the reference fiber 25 is embedded in the same house as the pressure sensor 23.

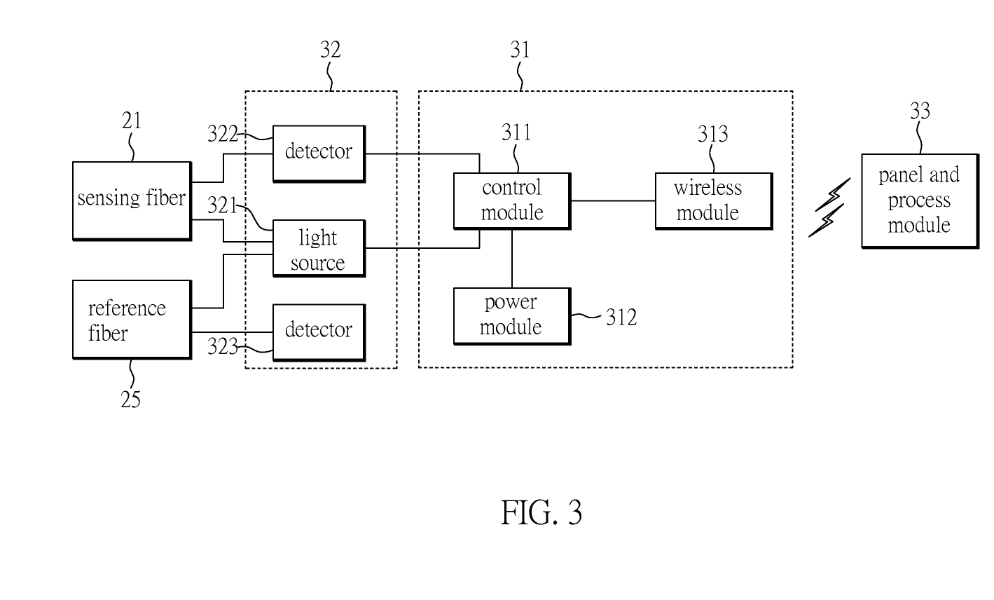

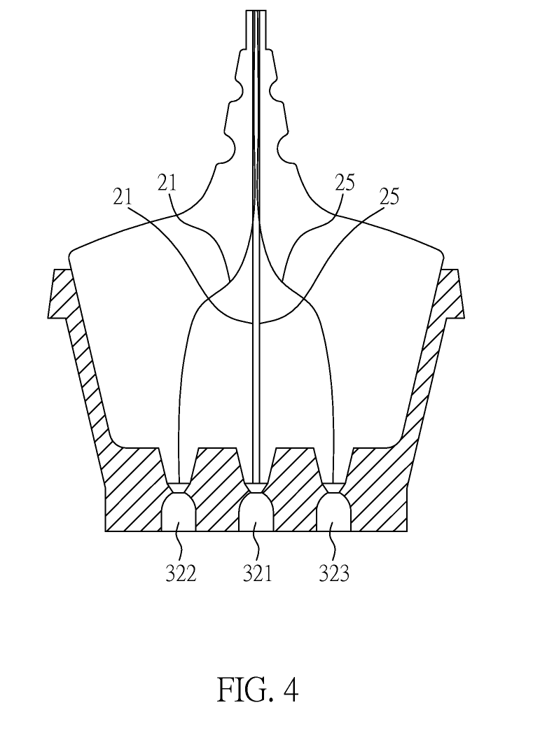

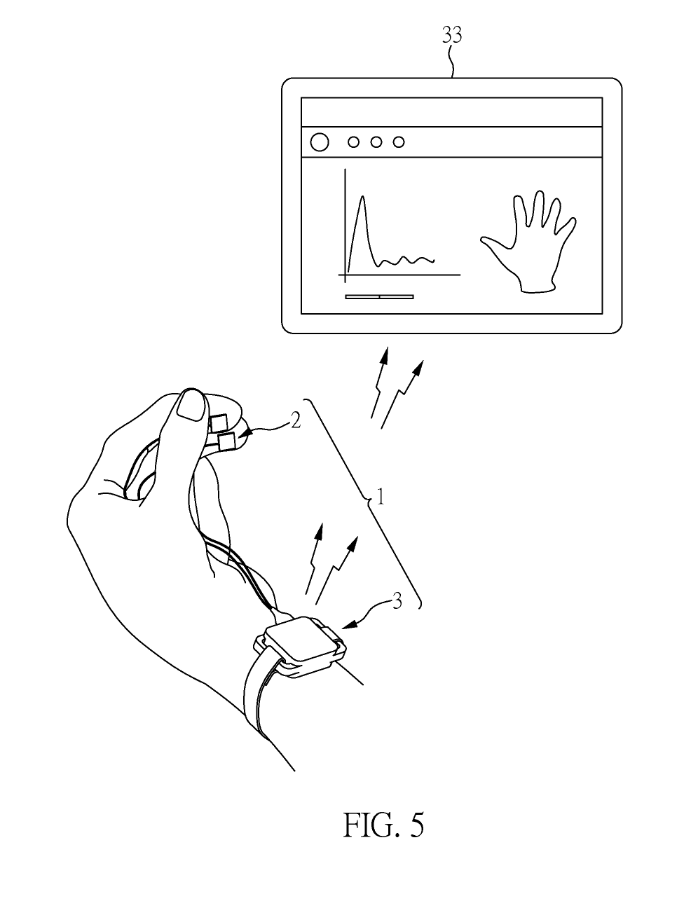

With reference to FIG. 3, FIG. 4 and FIG. 5, a systematic diagram of a palpation diagnostic device, a connecting schematic diagram of a sensing fiber and reference fiber and a schematic diagram of a palpation diagnostic device according to a preferred embodiment of the present invention. As shown in FIG. 3, the housing 31 is provided with a control module 311, a power module 312, and a wireless transmitting module 313, the connector 32 having a light source 321 and detectors 322 and 323. The control module 311 is electrically coupled to the power module 312, the wireless transmitting module 313, the light source 321, and the detector 322, while the light source 321 and the detector 322 are electrically coupled to the sensing fiber 21 respectively, the power module 312 providing electric power for the optical pressure sensor 2 and control device 3; and wherein, the light source 321 is arranged to send signal to the sensing fiber 21 and reference fiber 25, the detector 322 is arranged to receive the optical signal from sensing fiber 21, the detector 323 is arranged to receive the optical signal from reference fiber 25, and the control module 311 is arranged to receive the optical signals from both the detector 322 and 323 and control the signal therefrom. As shown in FIG. 4, the drawing shows how two connectors (male and female connectors) are connected. So a center port will represent the reference fiber 25 and sensing fiber 21 going into the light source 321 and the two on the side represent returning fiber going into two different detectors 322, 323. As shown in FIG. 5, the optical signal is transmitted to a panel and processing module 33 by the wireless transmitting module 313. Users can see the signal changes of every optical pressure sensor 2 through the panel and processing module 33 when using the palpation diagnostic device 1.

In the present embodiment, every optical pressure sensor 2 is deformed through the force applied on the holder 20, such that the intensity of the optical signal in the optical pressure sensor 2 is attenuated in response to the applied force. The sensing fiber 21 is a 250 μm bare plastic optical fiber with a 240 μm PMMA core and thin fluorinated polymer cladding.

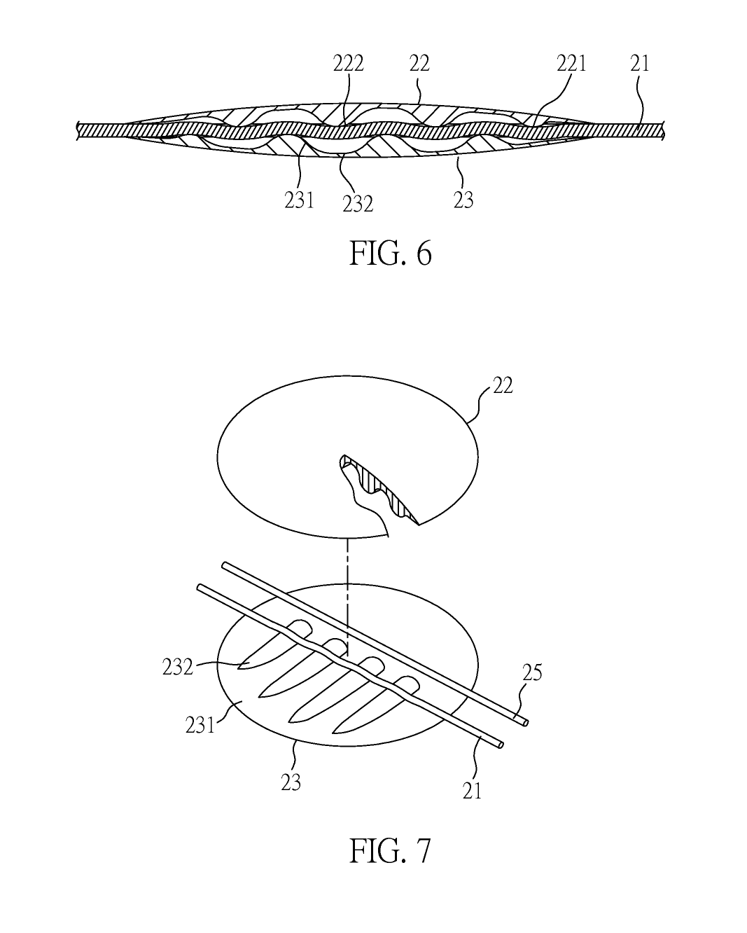

With reference to FIG. 6 and FIG. 7, a side sectional view and a schematic diagram of an optical pressure sensor according to a preferred embodiment of the present invention. As shown in FIG. 6 and FIG. 7, the holder 20 is a pad which comprises a first polymer patch 22 and a second polymer patch 23. The optical pressure sensor 2 is the optical fiber sensor which comprises a sensing fiber 21 embedded in between the first polymer patch 22 and the second polymer patch 23. Besides, the sensing fiber 21 is covered with an elastomer (not shown) which is filled between the first polymer patch 22 and the second polymer patch 23. Therefore, the sensing fiber 21 is slightly bent in accordance with the applied force applied on the first polymer patch 22, the intensity of the optical signal in the sensing fiber 21 is attenuated by slightly bending of the sensing fiber 21, and the attenuated intensity determines the value of the applied force on the first polymer patch 22 based on an attenuation of the intensity of the optical signal. The reference fiber 25 goes through the side of polymer patch 23 that doesn't have the teeth (notice only half of the patch includes the teeth). Therefore, the reference fiber 25 isn't bent when the patch is compressed, but fiber helps compensate the ambient noise received by the sensing fiber 21.

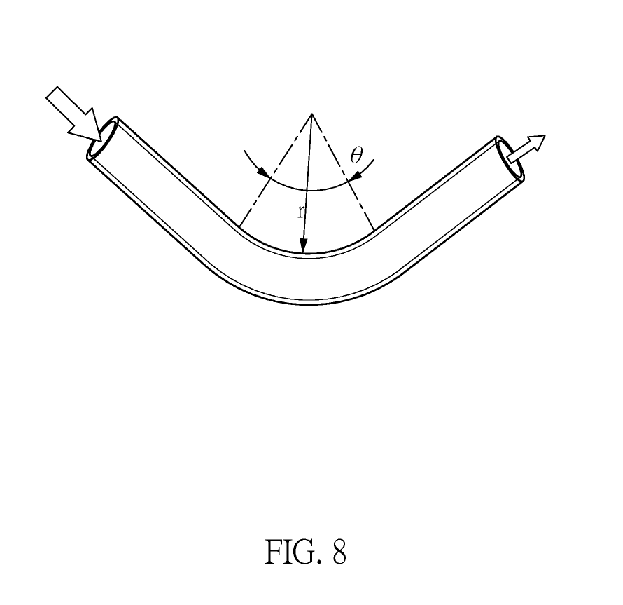

With reference to FIG. 8, the figure illustrates a micro-bend sensing fiber according to a preferred embodiment of the present invention. As shown in FIG. 8, fiber optic bendloss is a technique that has already been used in sensors for different applications. However, the attenuation of light through the sensing fiber 21 increases exponentially with the angle about which the fiber is bent (θ) and with a smaller bending radius (r). In the general simplified formula they obtained:

Lₜₒₜₐₗ=Ae^{Bθ-Cr}

where Lₜₒₜₐₗ is the total light loss in dB and A, B, and C are constants.

Meanwhile, referring to FIG. 6 and FIG. 7, in the present embodiment, the first polymer patch 22 comprises: a plurality of first teeth 222 disposed on a surface 221 of the first polymer patch 22, and a plurality of second teeth 232 disposed on a surface 231 of the second polymer patch 23 and engaged with the plurality of corresponding first teeth 222, sensing fiber 21 is disposed in between the plurality of corresponding first teeth 222 and the plurality of corresponding second teeth 232, and the sensing fiber 21 is in a series of corrugated shape by means of the plurality of first teeth 222 and the plurality of second teeth 232.

When no load is applied, the sensing fiber 21 is slightly pre-bent, bringing the light loss of the sensor into the highly sensitive range. When force is applied across the sensor, the plurality of corresponding first teeth 222 and the plurality of corresponding second teeth 232 are engaged with the sensing fiber 21 such that the sensing fiber 21 is induced with additional corrugation, resulting in a smaller bend radius and greater angle of bend for each tooth. Both of these factors result in light loss which is related to the force applied in a monotonic function as we mentioned before. This attenuation is proportional to the amount of bending the sensing fiber 21 is subjected to and can be related to the force applied. In this way, the optical signal passing through the sensing fiber 21 can be measured and calibrated into a real-time force measurement that is highly sensitive to manual forces applied by the hands.

In the present embodiment, the plurality of second teeth 232 of the second polymer patch 23 are engaged in the corresponding the plurality of first teeth 222, so the sensing fiber 21 is in a series of corrugated shape by means of the plurality of first teeth 222 and the plurality of second teeth 232 when the force is applied thereon. Therefore, when a force is applied on the top surface of the holder 20, the bending degree can be more obvious by matching the plurality of first teeth 222 and the plurality of second teeth 232, so that the variation of the amount of optical signal can be more sensitive and can provide precise and immediate information based on quantitative feedback for the users.

Furthermore, the first polymer patch 22 and the second polymer patch 23 are selected from a group consisting of a polymer, a plastic, a silicone rubber, polydimethylsiloxane (PDMS), elastomeric polymer containing polydimethylsiloxane (PDMS), or the combinations thereof. In the present embodiment, the first polymer patch 22 and the second polymer patch 23 are formed with plastic elastomers.

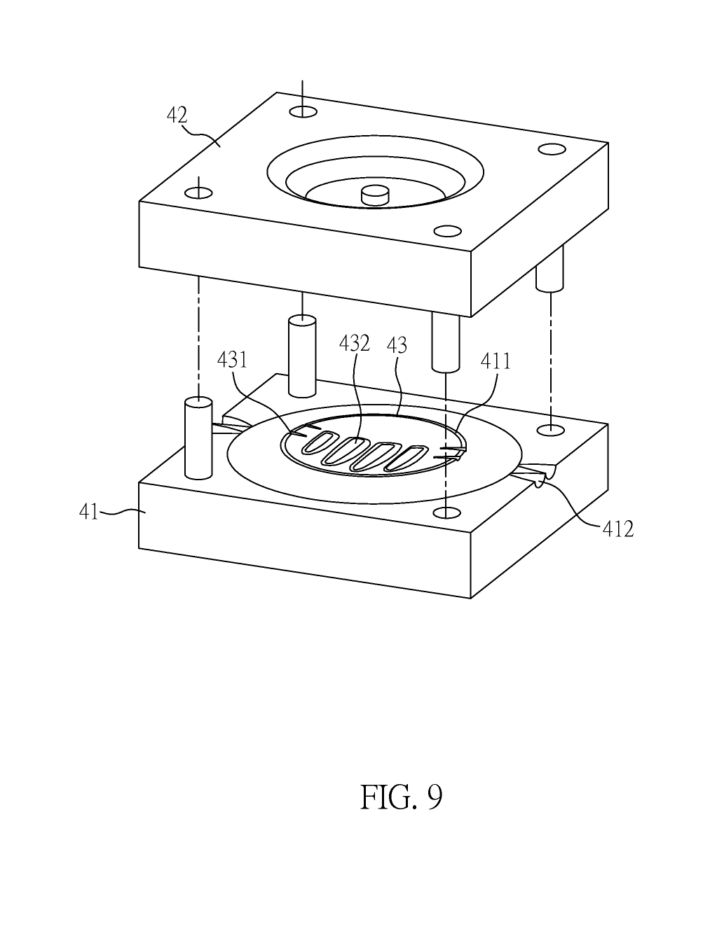

To achieve the geometric structure design of the optical pressure sensor 2, the first polymer patch 22 and the second polymer patch 23 are fabricated using 3D Polyjet. With reference to FIG. 9, a schematic diagram of a polymer patch manufacturing device according to a preferred embodiment of the present invention. As shown in FIG. 9, polymer patch manufacturing device includes two sets of molds 41, 42.

In the first stage of the process, liquid elastomer resin is dropped onto the convex cavity of the mold 41 to form a surface 411, about 3 drops and let sit until tacky. Beforehand, the mold 42 is sprayed with mold release, brushed to even the mold release residue, and let dry. It is then is pressed onto the mold 41, 42 and clamped until the resin cures so as to form a polymer patch. Excess resin overflows into a plurality of troughs 412 of mold 41 when pressed. When separated, a plurality of tooth surface 432 of the sensor is created by the imprint of the mold 42 which shape a surface 431 thereon. Therefore, the first polymer patch 22 and the second polymer patch 23 are fabricated by this method.

In the present embodiment, a core of the sensing fiber 21 is made by PMMA, and the cladding of the sensing fiber 21 is made by thin fluorinated polymer.

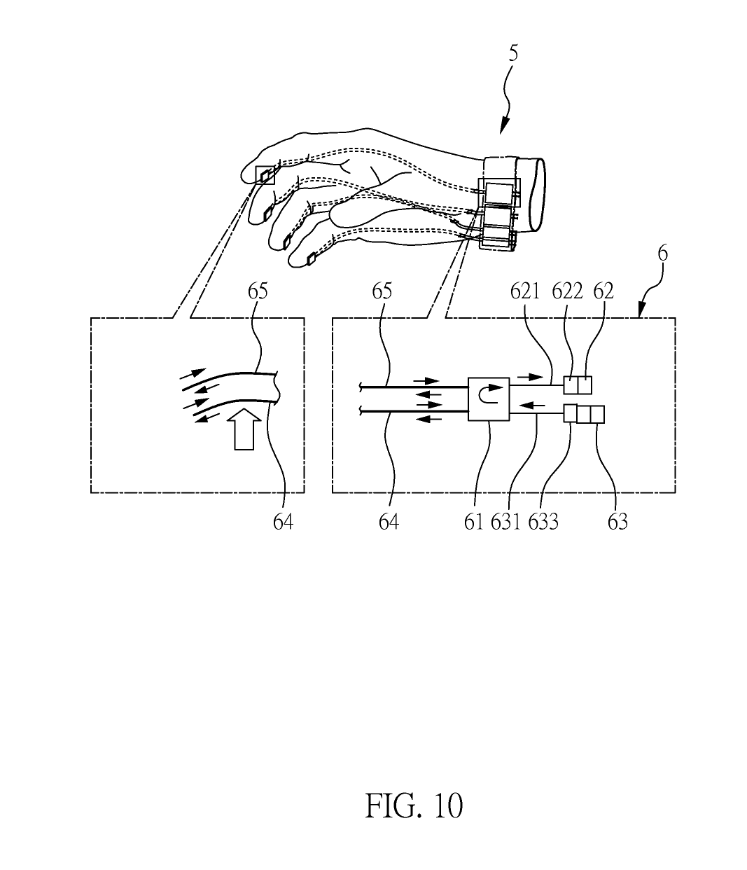

With reference to FIG. 10, a schematic diagram of a pressure sensing apparatus according to alternate preferred embodiment of the present invention. As shown in FIG. 10, a pressure sensing apparatus 5 comprising: at least one optical pressure sensor 6 embedded in a holder 60; wherein the optical pressure sensor 6 is an optical fiber sensor or a micro-fabricated waveguide sensor; the optical pressure sensor 6 is provided with a phase modulation, a micro-bend loss structure, or a macro-bend loss structure to perform quantitative sensing.

In the present embodiment, the optical pressure sensor 6 is an optical fiber sensor and is provided with a phase modulation, which comprises: a 2×2 coupler 61, a first sensing arm 64, a second sensing arm 65, a photodetector 62, and a light source 63, the 2×2 coupler 61 being coupled to the first sensing arm 64, the second sensing arm 65, the photodetector 62, and the light source 63 respectively.

In the present embodiment, the first sensing arm 64 and the second sensing arm 65 are fibers, having a metal deposited gold mirror (not shown) at the end point thereon to reflect the optical signal. The photodetector 62 and the light source 63 are electrically coupled to the 2×2 coupler 61 by using a fiber 621, 631. In addition, the photodetector 62 is coupled to a linear polarizer 622 which is in front of the photodetector 62; Laser diode is used as the monochromatic light source 63. And wherein, an optical signal emitted from the light source 63 becomes two input optical signals with same light intensity through the 2×2 coupler 61, the two input optical signals pass through the first sensing arm 64 and the second sensing arm 65 to both endpoints therein so as to become two reflected optical signals; and when the two reflected optical signals pass through the first sensing arm 64 and the second sensing arm 65 respectively to the photodetector by the 2×2 coupler 61, there is a phase shift between the two reflected optical signals so that it shall be coupled to form an interference pattern.

In the present embodiment, the optical pressure sensor 6 is provided with the Michelson interferometer configuration. The optical pressure sensor 6 utilizes the relative change in the optical path length between the first sensing arm 64 and the second sensing arm 65 due to an elongation or optical index change in the fiber, and the optical signal combined by the coupler 61 is provided with the light interferometric characteristics due to the relative change in the optical path length. When the first sensing arm 64 and the second sensing arm 65 are affected by applied force, Michelson interferometer configuration may increase the sensitivity of the optical pressure sensor 6, which can provide precise and immediate information based on quantitative feedback for the users. Furthermore, the holder 60 can be a pad or a glove. In the present embodiment, the holder 60 is a pad.

More precisely, based on the Michelson interferometer configuration in the optical pressure sensor 6, the bend induced phase shift may be written as

Δφ=kΔL+LΔk

Wherein, Δφ is affected by two conditions. The first term kΔL corresponds to the change in length of the first sensing arm 64 and the second sensing arm 65, and the second term LΔK to the photoelastic effect. When strain configuration is taken into account, the first term kΔL represents the effect of the physical change of length due to the strain becomes:

Δφ=kₒnS₁ΔL

where S is the strain vector and the subscript 1 of the strain vector refers to the longitudinal direction, i.e., along the axis of the first sensing arm 64 and the second sensing arm 65, in this case x direction. The transverse components 2 or 3 of the optical indicatrix are equivalent here because of the radial symmetry. Strain vector will be different depends on different in stress.

The second term, the change in phase due to a change in k, come about from two effects: the strain-optic effect whereby the strain changes the refractive index of the first sensing arm 64 and the second sensing arm 65, and a wave guide mode dispersion effect due to a change in diameter of the first sensing arm 64 and the second sensing arm 65 produced by strain:

$$L Δ k = L \frac{d k}{d n} Δ n + L \frac{d k}{d D} Δ D$$

The strain-optic effect whereby the strain changes the refractive index of the fiber when light is propagating in the axial direction (x direction) of the first sensing arm 64 and the second sensing arm 65 is expressed as:

$$Δ n = - \frac{1}{2} {n}^{3} {Δ ( {n}^{\frac{1}{2}} )}_{x , y , z}$$

Based on the theory, the propagation constant is k=nko, and hence

$$\frac{d k}{d n} = {k}_{o} .$$

The strain-optic effect appears as a change in the optical indicatrix

$${Δ ( \frac{1}{{n}^{2}} )}_{i} = {\sum }_{i = 1}^{6} {ρ}_{ij} {S}_{j}$$

where μ is the Poisson's ratio. The strain ε is related to the applied pressure P by the value of Young's modulus, E, in the form of ε=−P/E. Without shear strain, S₄, S₅, S₆=0, we only need to considered i, j=1, 2, 3 elements of the strain-optic sensor for a homogeneous isotropic material. For an isotropic medium, ρᵢⱼ has only two numerical values, designated ρ₁₁ and ρ₁₂

When plug in with values, the effect by the change in diameter is relatively small than the other two terms by two or three orders of magnitude. Therefore, the bend induced phase shift is reduced to length change and photoelastic effect

$$Δ φ = {k}_{o} {nS}_{1} Δ L - ( \frac{1}{2} ) {Lk}_{o} {n}^{3} {\sum }_{i = 1}^{6} {ρ}_{ij} {S}_{j}$$

Intensity received at detector 62,

$$\begin{matrix}I = < {E}_{r}^{2} > + < {E}_{s}^{2} > + 2 < {E}_{r} {E}_{s} > \\ = {I}_{r} + {I}_{s} + 2 {( {I}_{r} {I}_{s} )}^{0.5} cos ( Δ φ ) \\ = {I}_{o} [ {α}_{r} {k}_{f} {k}_{b} + {α}_{s} ( 1 - {k}_{f} ) + 2 \sqrt{{α}_{r} {α}_{s} {k}_{f} {k}_{b} ( 1 - {k}_{f} )} cos ( Δφ ) ]\end{matrix}$$

Where < > denote a time average over a period >2π/ω₀, αᵣ and αₛ are optical loss associate with reference and sensing paths (the first sensing arm 64 and the second sensing arm 65), and k_{f}, k_{b} are associate with coupling coefficients with light traveling forward toward and back from the sensing arms 64, 65.

Therefore, in the present embodiment, the optical pressure sensor 6 of the pressure sensing apparatus 5 is provided with the Michelson interferometer configuration having a way to measure the applied force quantitatively. In the present embodiment, light source 63 is a laser diode. In order to avoid the reflected light from 2×2 coupler 61 interfering with the input light, the combination of linear polarizer 633 and ¼ wave plate 633 are disposed to change the reflected light from linearly polarized to circularly polarized to the input channel.

Another way to avoid the reflected from 2×2 coupler 61 interfering with the input light is replacing the combination of linear polarizer 633 and ¼ wave plate 633 with a fiber optic isolator.

The force is measured based on the induced strain on the first sensing arm 64 and the second sensing arm 65. When users applies a force on the optical pressure sensor 6, the first sensing arm 64 and the second sensing arm 65 gets bent due to finger touching something, and a phase shift occurs between the two sensing arms 64, 65. The phase shift as described earlier will be proportional to the bending profile which in terms proportional to the applied force.

For small force, no applicator is needed and phase shift of the pressure sensing apparatus 5 will be kept at less than π/2 to keep the operation within the linear region. According to the phase shift, fringes in different light intensities of the optical interference will change thereby.

If larger force measurement is required, the pressure sensing apparatus 5 must operate at the nonlinear region. In the nonlinear range in which large perturbations force is applied, the output goes into the nonlinear range, thereby inducing fringes.

If applied force is too small to be detected without the applicator such as monitoring sound or heartbeat, as shown in FIG. 5 and FIG. 6, the holder 60 may include the first polymer patch 22 and the second polymer patch 23. The sensing arms 64, 65 are embedded in between the first polymer patch 22 and the second polymer patch 23, and are covered by the elastomer between the first polymer patch 22 and the second polymer patch 23. Furthermore, the first polymer patch 22 and the second polymer patch 23 are selected from a group consisting of a polymer, a plastic, a silicone rubber, polydimethylsiloxane (PDMS), an elastomeric polymer containing polydimethylsiloxane (PDMS), or combinations thereof. In the present embodiment, the first polymer patch 22 comprises a plurality of first teeth 222 disposed on a surface 221 of the first polymer patch 22 and a plurality of second teeth 232 disposed on a surface 231 of the second polymer patch 23 and engaged with the plurality of corresponding first teeth 222, and the sensing arms 64, 65 are a series of corrugated shape by means of the plurality of first teeth 222 and the plurality of second teeth 232. Thereby, using the structure as aforementioned can enhance the sensitivity of the sensing arms 64, 65.

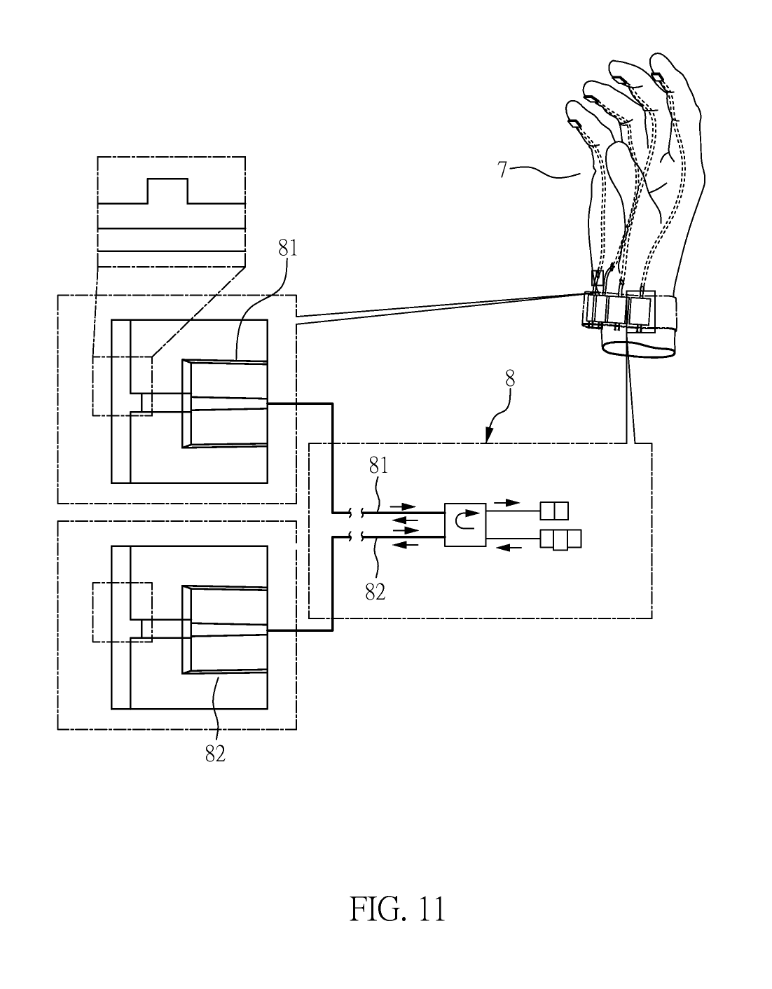

With reference to FIG. 11, a schematic diagram of a pressure sensing apparatus according to another alternate preferred embodiment of the present invention. As shown in FIG. 11, the difference between a pressure sensing apparatus 7 and the pressure sensing apparatus 5 shown in FIG. 10 is mainly focusing on: an optical pressure sensor 8 that the pressure sensing apparatus 7 comprises is a micro-fabricated waveguide sensor and also has Michelson interferometer configuration therein. A first sensing arm 81 and a second sensing arm 82 in the optical pressure sensor 8 are micro-fabricated waveguides. In the present embodiment, sensing endpoints of the first sensing arm 81 and the second sensing arm 82 may increase the sensitivity of the optical signal by using a plurality of polymer patch which a holder 80 includes.

Although the present invention has been explained in relation to its preferred embodiment, it is to be understood that many other possible modifications and variations can be made without departing from the spirit and scope of the invention as hereinafter claimed.

What is claimed is:

1. A palpation diagnostic device, comprising: an optical pressure sensor embedded in a holder;
wherein the optical pressure sensor is an optical fiber sensor, or a micro-fabricated waveguide sensor to be disposed on a finger or a palm; and
the optical pressure sensor is configured to receive an optical signal whose intensity is attenuated when a force is applied on the optical pressure sensors.

2. The palpation diagnostic device as claimed in claim 1, wherein the optical pressure sensor is deformed through the force applied on the holder such that the intensity of the optical signal in the optical pressure sensor is attenuated in response to the applied force.

3. The palpation diagnostic device as claimed in claim 2, wherein the holder is a pad which comprises a first polymer patch and a second polymer patch.

4. The palpation diagnostic device as claimed in claim 3, wherein the optical pressure sensor is the optical fiber sensor which comprises a sensing fiber and a reference fiber both embedded in between the first polymer patch and the second polymer patch.

5. The palpation diagnostic device as claimed in claim 4, wherein the sensing fiber is slightly bent in accordance with the applied force applied on the first polymer patch, the intensity of the optical signal in the sensing fiber is attenuated by slightly bending of the sensing fiber, and the attenuated intensity determines the value of the applied force on the first polymer patch based on an attenuation of the intensity of the optical signal.

6. The palpation diagnostic device as claimed in claim 4, wherein the reference fiber isn't bent when the applied force applied on the first polymer patch, but the reference fiber helps compensate the ambient noise and temperature change received by the sensing fiber.

7. The palpation diagnostic device as claimed in claim 4, wherein the first polymer patch comprises: a plurality of first teeth disposed on a surface of the first polymer patch, and a plurality of second teeth disposed on a surface of the second polymer patch and engaged with the plurality of corresponding first teeth, and the sensing fiber is in a series of corrugated shape by means of the plurality of first teeth and the plurality of second teeth, while the reference fiber goes through the side that doesn't have the teeth in the first and second polymer patches

8. The palpation diagnostic device as claimed in claim 3, wherein the sensing fiber is covered by an elastomer between the first polymer patch and the second polymer patch.

9. The palpation diagnostic device as claimed in claim 3, wherein the first polymer patch and the second polymer patch are selected from a group consisting of a polymer, a plastic, a silicone rubber, polydimethylsiloxane (PDMS), elastomeric polymer containing polydimethylsiloxane (PDMS), or the combinations thereof.

10. The palpation diagnostic device as claimed in claim 4, further comprising a control device, wherein the control device is electrically coupled to the optical pressure sensor.

11. The palpation diagnostic device as claimed in claim 10, wherein the control device comprises a control module, a light source, and a photodetector;
the control module is electrically coupled to the light source and photodetectors, the light source and the photodetectors are electrically coupled to the sensing fiber and reference fiber respectively; and the light source is arranged to emit the optical signal to the sensing fiber and the reference fiber, the photodetectors are arranged to receive the optical signals from the sensing fiber and reference fiber, and the control module is arranged to receive the optical signal from the photodetectors and process the optical signal therein.

12. A pressure sensing apparatus, comprising: an optical pressure sensor embedded in a holder;
wherein the optical pressure sensor is an optical fiber sensor or a micro-fabricated waveguide sensor;
the optical pressure sensor is provided with a phase modulation, a micro-bend loss structure, or a macro-bend loss structure to perform quantitative sensing.

13. The pressure sensing apparatus as claimed in claim 12, wherein the holder is a pad which comprises a first polymer patch and a second polymer patch.

14. The pressure sensing apparatus as claimed in claim 12, wherein optical pressure sensor is provided with the Michelson interferometer configuration which comprises: a 2×2 coupler, a first sensing arm, a second sensing arm, a photodetector, and a light source;
the 2×2 coupler being coupled to the first sensing arm, second sensing arm, the photodetector, and the light source respectively; an optical signal emitted from the light source becomes two input optical signals with same light intensity through the 2×2 coupler, the two input optical signals pass through the first sensing arm and the second sensing arm to both endpoints therein so as to become two reflected optical signals; and when the two reflected optical signals pass through the first sensing arm and the second sensing arm respectively to the photodetector by the 2×2 coupler, there is a phase shift between the two reflected optical signals so that it shall be coupled to form an interference pattern.

15. The pressure sensing apparatus as claimed in claim 14, wherein when the first sensing arm and second sensing arm are bent by the applied force, the phase shift will be changed in accordance with the bending level of the first sensing arm and second sensing arm.

16. The pressure sensing apparatus as claimed in claim 15, wherein the pressure sensing apparatus is operated in a linear region when the phase shift imposed by the applied force is lower than π/2, and the light intensity of the interference pattern will be changed in accordance with the phase shift.

17. The pressure sensing apparatus as claimed in claim 15, wherein the pressure sensing apparatus is operated in a nonlinear region and is provided with the plurality of interference pattern of interference fringe when the phase shift imposed by the applied force is upper than π/2.

18. The pressure sensing apparatus as claimed in claim 14, wherein the sensing arms are embedded in between the first polymer patch and the second polymer patch.

19. The pressure sensing apparatus as claimed in claim 16, wherein the sensing arms are covered by the elastomer between the first polymer patch and the second polymer patch.

20. The pressure sensing apparatus as claimed in claim 14, wherein the first polymer patch comprises a plurality of first teeth disposed on a surface of the first polymer patch and a plurality of second teeth disposed on a surface of the second polymer patch and engaged with the plurality of corresponding first teeth, and the sensing fiber is a series of corrugated shape by means of the plurality of first teeth and the plurality of second teeth.

21. The pressure sensing apparatus as claimed in claim 14, wherein the first polymer patch and the second polymer patch are selected from a group consisting of a polymer, a plastic, a silicone rubber, polydimethylsiloxane (PDMS), an elastomeric polymer containing polydimethylsiloxane (PDMS), or combinations thereof.
